(19) United States
(12) Patent Application Publication
(10) Pub. No.: US 2024/0019426 A1
(43) Pub. Date: Jan. 18, 2024

(54) Lateral Flow Devices for High Sensitivity Detection of Coronavirus Infection, and Methods of Making and Using the Same

(71) Applicant: Sorrento Therapeutics, Inc., San Diego, CA (US)

(72) Inventor: Henry Hongjun Ji, Rancho Santa Fe, CA (US)

(73) Assignee: Sorrento Therapeutics, Inc., San Diego, CA (US)

(21) Appl. No.: 18/251,673

(22) PCT Filed: Nov. 3, 2021
(86) PCT No.: PCT/US2021/057932

§ 371 Date: May 3, 2023

Related U.S. Application Data
(60) Provisional application No. 63/109,859, filed on Nov. 4, 2020.

(51) Int. Cl. G01N 33/543; B01L 3/00

(52) U.S. Cl. CPC G01N 33/54388; B01L 3/5023; B01L 2400/0406; B01L 2200/143; B01L 2300/069; B01L 2200/16

(57) ABSTRACT

The present disclosure provides, inter alia, a lateral flow device, and methods of use of the device, for accurately and rapidly detecting the presence of a coronavirus or a coronavirus infection in a subject, such as a SARS-CoV-2 virus or virus infection. The lateral flow device detects in a sample from the subject the presence or absence of a coronavirus protein (e.g., a SARS-CoV-2 protein), such as an S protein, an Si protein, or nucleocapsid protein. The lateral flow device comprises, for example, Pt or Au/Pt nanoparticle-antibody conjugates for detection of coronavirus protein or coronavirus infection, such as a SARS-CoV-2 protein or a SARS-CoV-2 infection.

### CROSS REFERENCE TO RELATED APPLICATIONS

This application claims priority to U.S. provisional patent application No. 63/109,859, filed on Nov. 4, 2020, the content and disclosure of which is incorporated by reference in its entirety for all purposes.

### BACKGROUND

Severe acute respiratory syndrome coronavirus 2 (SARS-CoV-2) and associated variants, the viruses that cause COVID-19, was first reported in late 2019 in Wuhan China and has since spread throughout the world. Rapid and accurate detection of a coronavirus antigen from a sample such as a nasal swab sample can be critical to curbing the spread of the pandemic. Sensitive techniques to detect minute quantities of antigens, such as coronavirus antigens, from complex samples remains challenging. Such efforts have included development of technologies to amplify detection signals. However, despite the advances in the art, many pathogen detection strategies require reagents that are difficult or expensive to produce, or otherwise require equipment that is not amenable to point of care or field monitoring. There is an urgent need for antigen detection reagents that can accurately, inexpensively, and rapidly provide detectable signals for very low levels of antigens of interest from complex samples such as throat or nasal swabs. The present disclosure addresses, inter alia, these and related needs.

### SUMMARY

The disclosure provided herein and throughout is directed to, inter alia, lateral flow devices for accurately and rapidly detecting the presence of coronavirus or a coronavirus infection, such as a SARS-Cov-2 virus or a SARS-Cov-2 virus infection, in a subject, and method of using such devices. In certain embodiments, which may be combined with other embodiments disclosed herein and throughout, the lateral flow devices, for example, detect, the presence of at least one SARS-CoV-2 antigen, such as but not limited to the nucleocapsid protein of SARS-CoV-2 (Genbank Accession No. YP_009724397). In certain embodiments, which may be combined with other embodiments disclosed herein and throughout, the lateral flow device comprises a test strip comprising, in the following order: a sample application zone, a conjugate pad, an antigen detection zone, and, preferably, a control detection zone. In certain embodiments, which may be combined with other embodiments disclosed herein and throughout, the test strip preferably also comprises, at the end opposite to the end having the sample application zone, an absorbent pad to drive the flow of sample and reagents from the sample application zone to the detection zones.

In certain embodiments, which may be combined with other embodiments disclosed herein and throughout, the sample application zone comprises absorbent material and can optionally include reagents for improving or maintaining the solubility of sample components such as proteins. The sample pad in some embodiments can also serve as a filter for retaining any particles, cells, broken cells, aggregates, and other relatively large particles in the sample application zone.

In certain embodiments, which may be combined with other embodiments disclosed herein and throughout, the conjugate pad comprises an absorbent material and includes conjugates that, as disclosed herein, comprise an antibody that specifically binds a SARS-CoV-2 antigen, such as but not limited to an S protein or a nucleocapsid protein, where the antibody is conjugated to one or more nanoparticles for detection of the formation or presence of an antigen-antibody conjugate. In certain embodiments, which may be combined with other embodiments disclosed herein and throughout, the nanoparticles comprise platinum, and can comprise, for example, colloidal gold/platinum particles, or can comprise, as nonlimiting examples, a bimetallic nanoparticle such as a platinum (Pt)-palladium (Pd) bimetallic nanoparticle (Pt/Pd NP), a platinum (Pt)-cobalt (Co) bimetallic nanoparticle (Pt/Co NP), a platinum (Pt)-nickel (Ni) bimetallic nanoparticle (Pt/Ni NP), a platinum (Pt)-iron (Fe) bimetallic nanoparticle (Pt/Fe NP), or a is a platinum (Pt)-gold (Au) bimetallic nanoparticle (Pt/Au NP). Such bimetallic platinum-including nanoparticles have peroxidase activity. In certain embodiments, which may be combined with other embodiments disclosed herein and throughout, the nanoparticles comprise gold-platinum nanoparticles. Multiple nanoparticles can be conjugated to a single antibody, such for example, an antibody that binds the nucleocapsid protein of SARS-CoV-2 or the S protein of SARS-CoV-2. Thus, in certain embodiments, which may be combined with other embodiments disclosed herein and throughout, the presence of the bimetallic NPs is detected by their catalytic action on an appropriate substrate, such as but not limited to those disclosed below.

In certain embodiments, which may be combined with other embodiments disclosed herein and throughout, Pt based bi-metallic NPs have a high surface area and can demonstrate excellent catalytic performance including under conditions in which the activity of enzymatic catalysts would be inhibited.

In certain embodiments, which may be combined with other embodiments disclosed herein and throughout, the antigen detection zone of the test strip comprises a lateral flow membrane comprising an absorbent material which includes a test line, wherein the test line includes pre-set immobilized capture reagents that specifically bind the antigen in a noncompetitive way with respect to the binding of the antibody-label conjugates (e.g., the anti-nucleocapsid protein antibody-Au/Pt NP conjugates) such sandwich type complexes are formed at the test line of immobilized antibody-antigen-labeled antibody. In certain embodiments, which may be combined with other embodiments disclosed herein and throughout, the control detection zone includes and a control line comprising pre-set immobilized capture reagents for binding antibody-label conjugates.

In certain embodiments, which may be combined with other embodiments disclosed herein and throughout, the test line can optionally further include a substrate for peroxidase activity that provides a quantifiable color change, such as, for example, the chromatographic compound 3,3′,5,5′-tetramethylbenzidine (TMB), aminoethyl carbazole (AEC), 3,3′-diaminobenzidine (DAB), or o-phenylenediamine dihydrochloride (oPD). Alternatively, the test strip may not include a chromogenic substrate. In certain embodiments where the test strip does not include a chromogenic peroxidase substrate, a substrate may be provided with the substrate, such as in a kit, for application to the substrate at a time period following sample application.

In certain embodiments, which may be combined with other embodiments disclosed herein and throughout, the absorbent pad at the end of the test strip opposite to the end where sample is dispensed comprises a second absorbent material that draws the liquid sample through the test strip.

In certain embodiments, which may be combined with other embodiments disclosed herein and throughout, the lateral flow membrane is in fluid communication with the absorbent pad, the sample application zone is in fluid communication with the conjugate pad, and the conjugate pad is in fluid communication with the lateral flow membrane.

In certain embodiments, which may be combined with other embodiments disclosed herein and throughout, provided are lateral flow devices comprising a test strip, wherein the test strip comprises: a sample application zone, a conjugate pad, an antigen detection zone comprising a test line and a control line; and an absorbent pad to drive the flow of sample and reagents from the sample application zone to the detection zones. In certain embodiments, which may be combined with other embodiments disclosed herein and throughout, the sample application zone comprises absorbent material and can optionally include reagents for improving or maintaining the solubility of sample components such as proteins.

In certain embodiments, which may be combined with other embodiments disclosed herein and throughout, the conjugate pad comprises conjugates that comprise an antibody that specifically binds a SARS-CoV-2 antigen.

In certain embodiments, which may be combined with other embodiments disclosed herein and throughout, the conjugate pad comprises conjugates that comprise an antibody that specifically binds a SARS-CoV-2 antigen, wherein the SARS-CoV-2 antigen is an S protein or a nucleocapsid protein.

In certain embodiments, which may be combined with other embodiments disclosed herein and throughout, the conjugate pad comprises conjugates that comprise an antibody that is conjugated to a nanoparticle for detection of an antigen-antibody conjugate.

In certain embodiments, which may be combined with other embodiments disclosed herein and throughout, the conjugate pad comprises conjugates that comprise an antibody conjugated to a nanoparticle for detection of an antigen-antibody conjugate, wherein the nanoparticle comprises platinum.

In certain embodiments, which may be combined with other embodiments disclosed herein and throughout, the conjugate pad comprises conjugates that comprise an antibody conjugated to a nanoparticle for detection of an antigen-antibody conjugate, wherein the nanoparticle comprises colloidal gold/platinum particles.

In certain embodiments, which may be combined with other embodiments disclosed herein and throughout, the conjugate pad comprises conjugates that comprise an antibody conjugated to a nanoparticle for detection of an antigen-antibody conjugate, wherein the nanoparticle comprises a bimetallic nanoparticle.

In certain embodiments, which may be combined with other embodiments disclosed herein and throughout, the conjugate pad comprises conjugates that comprise an antibody conjugated to a nanoparticle for detection of an antigen-antibody conjugate, wherein the nanoparticle comprises a platinum (Pt)-palladium (Pd) bimetallic nanoparticle (Pt/Pd NP), a platinum (Pt)-cobalt (Co) bimetallic nanoparticle (Pt/Co NP), a platinum (Pt)-nickel (Ni) bimetallic nanoparticle (Pt/Ni NP), a platinum (Pt)-iron (Fe) bimetallic nanoparticle (Pt/Fe NP), or a platinum (Pt)-gold (Au) bimetallic nanoparticle (Pt/Au NP).

In certain embodiments, which may be combined with other embodiments disclosed herein and throughout, the conjugate pad comprises conjugates that comprise an antibody conjugated to a nanoparticle for detection of an antigen-antibody conjugate, wherein the nanoparticle has peroxidase activity.

In certain embodiments, which may be combined with other embodiments disclosed herein and throughout, the test line comprises pre-set immobilized capture antibodies.

In certain embodiments, which may be combined with other embodiments disclosed herein and throughout, the test line comprises a substrate for peroxidase activity that provides a quantifiable color change.

In certain embodiments, which may be combined with other embodiments disclosed herein and throughout, the test line comprises a substrate for peroxidase activity, wherein the substrate for peroxidase activity is 3,3′,5,5′-tetramethylbenzidine (TMB), aminoethyl carbazole (AEC), 3,3′-diaminobenzidine (DAB), or o-phenylenediamine dihydrochloride (oPD).

In certain embodiments, which may be combined with other embodiments disclosed herein and throughout, the lateral flow device has a sensitivity that is increased least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that does not comprise an antibody-Pt conjugate, an antibody-Au/Pt conjugate, or a platinum colloid core.

In certain embodiments, which may be combined with other embodiments disclosed herein and throughout, the lateral flow device has a sensitivity that is increased least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that comprises colloidal gold without platinum.

In certain embodiments, which may be combined with other embodiments disclosed herein and throughout, the lateral flow device has a sensitivity that is least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least at least 40-fold, at least 45-fold, at least 50-fold, at least 55-fold, at least 60-fold, at least 65-fold, at least 70-fold, at least 75-fold, at least 80-fold, at least 85-fold, at least 90-fold, at least 95-fold, at least 100-fold, or greater than one or more of QuickVue™, Ellume™ BD Veritor System™, BinaxNow™, PanBio™, and AccessBio™.

In certain embodiments, which may be combined with other embodiments disclosed herein and throughout, the lateral flow device has a specificity that is increased least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that does not comprise an antibody-Pt conjugate, an antibody-Au/Pt conjugate, or a platinum colloid core.

In certain embodiments, which may be combined with other embodiments disclosed herein and throughout, the lateral flow device has a specificity that is increased least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that comprises colloidal gold without platinum.

In certain embodiments, which may be combined with other embodiments disclosed herein and throughout, the lateral flow device has a specificity that is least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least at least 40-fold, at least 45-fold, at least 50-fold, at least 55-fold, at least 60-fold, at least 65-fold, at least 70-fold, at least 75-fold, at least 80-fold, at least 85-fold, at least 90-fold, at least 95-fold, at least 100-fold, or greater than one or more of QuickVue™, Ellume™ BD Veritor System™, BinaxNow™, PanBio™, and AccessBio™.

In certain embodiments, which may be combined with other embodiments disclosed herein and throughout, the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least lower, at least 40-fold lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least lower, at least 100-fold lower, or less than a lateral flow device and/or an assay that does not comprise an antibody-Pt conjugate, an antibody-Au/Pt conjugate, or a platinum colloid core (PtC).

In certain embodiments, which may be combined with other embodiments disclosed herein and throughout, the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least lower, at least 40-fold lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least lower, at least 100-fold lower, or less than a lateral flow device and/or an assay that comprises colloidal gold without platinum.

In certain embodiments, which may be combined with other embodiments disclosed herein and throughout, the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least lower, at least 40-fold lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least lower, at least 100-fold lower, or less than one or more of QuickVue™, Ellume™, BD Veritor System™, BinaxNow™, PanBio™, and AccessBio™ detection devices and/or assays.

In certain embodiments, which may be combined with other embodiments disclosed herein and throughout, provided are lateral flow devices that comprise a plurality of lateral flow regions arranged in the order:
a) a sample application zone for dispensing a liquid sample thereupon wherein the sample application zone comprises an absorbent material;
b) a conjugate pad comprising an absorbent material and pre-set conjugates which include (i) SARS-CoV-2 anti-nucleocapsid protein antibodies conjugated to platinum-containing nanoparticles,
c) a detection zone comprising a lateral flow membrane comprising an absorbent material which includes a test line and a control line, where the test line includes (i) a pre-set immobilized nucleocapsid protein capture reagents, and (ii) a control line comprising pre-set immobilized capture reagents that bind the anti-nucleocapsid protein antibody-platinum nanoparticle conjugates, and
d) an absorbent pad comprising an absorbent material,
where the lateral flow membrane is in fluid communication with the absorbent pad, the sample application zone is in fluid communication with the conjugate pad, and the conjugate pad is in fluid communication with the lateral flow membrane.

In certain embodiments, which may be combined with other embodiments disclosed herein and throughout, the lateral flow devices can be disposed in a housing which includes a base, a lid, two end walls and two side walls. The lid of the housing can include a cut-out region at the position of the sample port or sample pad for liquid sample dispensing, and the lid can further include a second cut-out region at the detection zone for use as an observation window.

In certain embodiments, which may be combined with other embodiments disclosed herein and throughout, kits are provided comprising a lateral flow device and any of the following: one or more sample tubes, one or more sample pouches, one or more swabs, and one or more peroxidase substrates. The kit can also further comprise a tube, pouch, or vial that includes a sample buffer for extracting test material from the swab after taking the sample from the subject. The tube, pouch or vial may include a lid that is connected to a tube or dropper for distributing sample from the tube, pouch, or vial to the test strip. Alternatively, the kit can further include a separate dropper or syringe-type device for removing sample from the tube, pouch, or vial and distributing the sample on the test strip. A tube or dropper can include markings, such as volumetric markings.

In certain embodiments, which may be combined with other embodiments disclosed herein and throughout, a kit comprising a lateral flow device as disclosed herein and throughout and one or more of: one or more swabs, one or more pouches or bottles comprising a sample buffer, one or more sample tubes, one or more droppers, and one or more pipetting devices. In certain embodiments, which may be combined with other embodiments disclosed herein and throughout, the kit can optionally further include one or more substrate solutions, e.g., a solution that includes a substrate that can be acted on by a peroxidase.

In certain embodiments, which may be combined with other embodiments disclosed herein and throughout, a kit comprises a lateral flow device as disclosed herein and throughout and one or more of: one or more swabs, one or more pouches or bottles comprising a sample buffer, one or more sample tubes, one or more droppers, one or more pipetting devices, and a colorimetric substrate for peroxidase activity.

In certain embodiments, which may be combined with other embodiments disclosed herein and throughout, a lateral flow device or kit has a test line comprising pre-set immobilized human nucleocapsid protein capture reagents, e.g., nucleocapsid protein antibodies, and the control line can comprise pre-set immobilized anti-IgG capture reagents.

In certain embodiments, which may be combined with other embodiments disclosed herein and throughout, a lateral flow device or kit includes a sample application zone that comprises a sample pad and a sample port, where the sample port is in fluid communication with the sample pad.

In certain embodiments, which may be combined with other embodiments disclosed herein and throughout, a lateral flow device or kit as provided herein can include antibody-Au/Pt nanoparticle (NP) conjugates.

In certain embodiments, which may be combined with other embodiments disclosed herein and throughout, the lateral flow device or kit comprises capture reagents (e.g., in the test line or control line) that bind biotin comprise avidin, streptavidin, NEUTRAVIDIN, EXTRAVIDIN, CAPTAVIDIN, or NEUTRALITE AVIDIN, or a truncated form thereof that retains biotin-binding activity, optionally wherein the avidin, streptavidin, NEUTRAVIDIN, EXTRAVIDIN, CAPTAVIDIN, or NEUTRALITE AVIDIN is optionally glycosylated.

In certain embodiments, which may be combined with other embodiments disclosed herein and throughout, are provided methods for detecting in a liquid sample from a subject the presence or absence of a SARS-Cov-2 protein, the methods comprising the steps of:
a) providing a liquid sample from the subject;
b) dispensing the liquid sample onto the sample application zone of a lateral flow device of any one of the embodiments provided herein under a condition that is suitable for lateral flow of the liquid sample and soluble proteins contained therein, wherein the lateral flow moves the liquid sample from the sample application zone through the conjugate pad of a test strip, through the detection zone, and through the absorbent pad, where a SARS-CoV-2 protein, such as a nucleocapsid protein, present in the sample may bind to Au/Pt nanoparticle antibody conjugates in the conjugate pad to form nucleocapsid protein-Au/Pt nanoparticle antibody conjugates complexes, and wherein the nucleocapsid protein-Au/Pt nanoparticle antibody conjugates complexes are able to migrate to the test line of the test strip, where the nucleocapsid protein—Au/Pt nanoparticle antibody conjugates may be detected. The method may further include: c) detecting a signal at the control line, adding a substrate for a peroxidase at the test line, and d) observing a colorimetric reaction at the test line.

In certain embodiments, which may be combined with other embodiments disclosed herein and throughout, are provided methods for detecting in a liquid sample from a subject the presence or absence of a SARS-Cov-2 protein, the methods comprising the steps of:
a) providing a liquid sample from the subject;
b) dispensing the liquid sample onto the sample application zone of a lateral flow device of any one of the embodiments provided herein under a condition that is suitable for lateral flow of the liquid sample and soluble proteins contained therein, wherein the lateral flow moves the liquid sample from the sample application zone through the conjugate pad of a test strip, through the detection zone, and through the absorbent pad, where a SARS-CoV-2 protein, such as a nucleocapsid protein, present in the sample may bind to Au/Pt nanoparticle antibody conjugates in the conjugate pad to form nucleocapsid protein-Au/Pt nanoparticle antibody conjugates complexes, and wherein the nucleocapsid protein-Au/Pt nanoparticle antibody conjugates complexes are able to migrate to the test line of the test strip, where the nucleocapsid protein—Au/Pt nanoparticle antibody conjugates may be detected. The method may further include: c) detecting a signal at the control line, adding a substrate for a peroxidase at the test line, and d) observing a colorimetric reaction at the test line, wherein the SARS-Cov-2 protein is a nucleocapsid (N) protein.

### DETAILED DESCRIPTION OF CERTAIN EXEMPLARY EMBODIMENTS

### Definitions

Unless defined otherwise, technical, and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975.

All of the references cited throughout this application are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. Alternatively, “about” or “comprising essentially of” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. These terms encompass native and artificial proteins, protein fragments and polypeptide analogs (such as muteins, variants, chimeric proteins, and fusion proteins) of a protein sequence as well as post-translationally, or otherwise covalently or non-covalently, modified proteins.

The term “mutation”, “modification”, or “variation”, or related terms, refers to a change in a nucleic acid sequence or amino acid sequence that differs from a reference nucleic acid sequence or a reference amino acid sequence, respectively. Examples of mutations includes a point mutation, insertion, deletion, amino acid substitution, inversion, rearrangement, splice, sequence fusion (e.g., gene fusion or RNA fusion), truncation, transversion, translocation, non-sense mutation, sequence repeat, single nucleotide polymorphism (SNP), or other genetic rearrangement.

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers in some embodiments to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrophotometry.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5′ Ed., US Dept. of Health and Human Services, PHS, NIH, NIH Publication no. 91-3242, 1991. Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof (or an antigen binding fragment thereof) that binds specifically to an antigen. Antigen binding portions (or the antigen binding fragment) may be produced by recombinant DNA techniques or by enzymatic or chemical cleavage of intact antibodies. Antigen binding portions (or antigen binding fragments) include, inter alia, Fab, Fab′, F(ab′)2, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations.

A “neutralizing antibody” and related terms refers to an antibody that is capable of specifically binding to the neutralizing epitope of its target antigen (e.g., coronavirus spike protein) and substantially inhibiting or eliminating the biological activity of the target antigen (e.g., coronavirus spike protein). The neutralizing antibody can reduce the biological activity of the target antigen by at least 20%, 30%, 40%, 50%, 60%, 70%, 80%, 90% or higher levels of reduced biological activity.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less.

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or VL domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989).

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen).

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

The term “Fc” or “Fc region” as used herein refers to the portion of an antibody heavy chain constant region beginning in or after the hinge region and ending at the C-terminus of the heavy chain. The Fc region comprises at least a portion of the CH and CH3 regions and may, or may not, include a portion of the hinge region. Two polypeptide chains each carrying a half Fc region can dimerize to form a full Fc domain. An Fc domain can bind Fc cell surface receptors and some proteins of the immune complement system. An Fc domain exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc domain can bind an Fc receptor, including FcγRI (e.g., CD64), FcγRII (e.g, CD32) and/or FcγRIII (e.g., CD16a).

The terms “labeled,” “detectably labeled,” or related terms as used herein refers to the presence of a detectable label or moiety for detection, e.g., wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens).

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters.

In one embodiment, the amino acid sequence of a test antibody may be similar but not identical to any of the amino acid sequences of the polypeptides that make up any of the anti-S-protein antibodies, or antigen binding protein thereof, described herein. The similarities between the test antibody and the polypeptides can be at least 95%, or at or at least 96% identical, or at least 97% identical, or at least 98% identical, or at least 99% identical, to any of the polypeptides that make up any of the anti-spike protein antibodies, or antigen binding protein thereof, described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

Polypeptides of the present disclosure (e.g., antibodies and antigen binding proteins) can be produced using any methods known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are preferably at least 65% pure, at least 75% pure, at least 85% pure, more preferably at least 95% pure, and most preferably at least 98% pure. Regardless of the exact numerical value of the purity, the polypeptide is sufficiently pure for use as a pharmaceutical product.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. A preferred form of glycosylation is sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

The term “SARS-Cov-2”, “SARS-CoV2, and the like, used interchangeably throughout, refers to the wild-type SARS-CoV-2 virus (also known as the “Washington virus” or the “Wuhan virus”), as well as any of the genetic lineages or variants thereof, such as the alpha (“United Kingdom”), beta (“South Africa”), gamma (“Brazil”), delta (“India”), lambda (“Peru”), Mu (“Columbia”), Kappa, Iota, Eta, Epsilon, Theta, and/or Zeta, and any others that emerge and may cause COVID in infected subjects.

The term “COVID” refers to coronavirus disease and/or the clinical symptoms and/or conditions that characterize the disease.

The term “subject” as used herein refers to human and non-human animals, including vertebrates, mammals, and non-mammals. In one embodiment, the subject can be human, non-human primates, simian, ape, murine (e.g., mice and rats), bovine, porcine, equine, canine, feline, caprine, lupine, ranine or piscine.

The term “sample” as used herein refers to a biological sample from a negative control subject, or from a subject having or suspected of having had a coronavirus infection. Biological samples include blood, serum, plasma, whole blood, urine, nasal swab fluid, bronchoalveolar lavage (BAL) fluid, or cerebrospinal fluid (CSF). The blood sample can be obtained by fingerstick or from venous blood (whole blood, serum, or plasma).

The term “S1 spike” or related terms as use herein refers to an S1 subunit of a spike protein from SARS-Cov-2 virus.

The term “fluid communication” as used herein refers to various absorbent materials described herein that are used to make a lateral flow device, where the absorbent materials are configured with each other to facilitate migration of a liquid sample in a lateral or capillary flow. The absorbent materials can be configured in end-to-end fluid connection, top-to-bottom fluid connection, or overlapping fluid connection.

The term “limit of detection” (“LoD”, used interchangeably throughout) refers to the lowest analyte concentration (such as a SARS-CoV-2 protein, such as an S protein, an S1 protein, or a nucleoprotein) likely to be reliably distinguished from the limit of blank (LoB) and at which detection is feasible. The LoD is determined, for example, by utilizing both the measured LoB and test replicates of a sample known to contain a low concentration of analyte. The limit of detection may be quantifiably determined or qualitatively determined (such as, for example, by visual inspection of a color-based readout).

The term “limit of blank” (“LoB”, used interchangeably throughout) is the highest apparent analyte concentration expected to be found when replicates of a blank sample containing no analyte are tested. The limit of blank may be quantifiably determined or qualitatively determined (such as, for example, by visual inspection of a color-based readout).

The term “sensitivity” (also known as “true positive” or “true positive rate”, all of which may be used interchangeably throughout), as it relates to diagnostic testing and sample detection, refers to the proportion of positive sample test results out of those samples (or subjects from which the samples were obtained) that (or who) actually have item being tested (such as the presence of an analyte, a condition, or an infection).

The term “specificity” (also known as “true negative” or “true negative rate”, all of which may be used interchangeably throughout), as it relates to diagnostic testing and sample detection, refers to the proportion of negative sample test results out of those samples (or subjects from which the samples were obtained) that (or who) do not actually have the item being tested (such as the presence of an analyte, a condition, or an infection).

### Lateral Flow Device

The present disclosure provides, inter alia, a lateral flow device which is easy to use, requires a small volume of liquid sample from the subject to be tested, and gives visual results (e.g., colorimetric) that indicate if the subject is infected with SARS-Cov-2. In one embodiment, the lateral flow device gives a qualitative “yes” or “no” signal to indicate the presence or absence of a coronavirus protein in a liquid sample from a subject suspected of having or having had an infection from a coronavirus.

The present disclosure provides, inter alia, a lateral flow device, and methods of use of the device, for accurately and rapidly detecting a SARS-CoV-2 infection in a subject. In some embodiments, the lateral flow device detects the presence of a polypeptide such as the nucleocapsid protein or S protein from SARS-CoV-2. In some embodiments, the lateral flow device gives a color change to indicate the presence of a coronavirus protein. A color change at a test line indicates a positive result, while lack of a color change at a test line indicates a negative result. In various embodiments the sensitivity of the assay is increased by the use of antibody-Au/Pt conjugates in the conjugate pad, where the Pt (platinum) component can have a peroxidase activity that provides the positive signal. In some embodiments the nanoparticles have multiple platinum molecules.

In certain embodiments, the lateral flow device and/or the assay disclosed herein and throughout comprises an antibody-Pt conjugate or an antibody-Au/Pt conjugate. In certain embodiments, the lateral flow device and/or the assay disclosed herein and throughout comprises a platinum colloid core (PtC).

In certain embodiments, the sensitivity of lateral flow devises and/or the assays disclosed herein and throughout is increased at least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that does not comprise an antibody-Pt conjugate, an antibody-Au/Pt conjugate, or a platinum colloid core. (PtC). In certain embodiments disclosed herein and throughout, the sensitivity of such lateral flow devices and/or assays is increased at least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least at least 75-fold, at least 80-fold, at least 85-fold, at least 90-fold, at least 95-fold, at least 100-fold, or greater relative to a lateral flow device and/or an assay that comprises colloidal gold without platinum.

In certain embodiments, the lateral flow devices and/or the assays disclosed herein and throughout have a sensitivity that is least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least at least 75-fold, at least 80-fold, at least 85-fold, at least 90-fold, at least 95-fold, at least 100-fold, or greater than one or more of QuickVue™, Ellume™, BD Veritor System™, BinaxNow™, PanBio™, and AccessBio™ detection devices and/or assays.

In certain embodiments, the specificity of such lateral flow devices and/or assays disclosed herein and throughout is increased at least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that does not comprise an antibody-Pt conjugate, an antibody-Au/Pt conjugate, or a platinum colloid core (PtC). In certain embodiments disclosed herein and throughout, the specificity of such lateral flow devices and/or assays is increased at least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least at least 75-fold, at least 80-fold, at least 85-fold, at least 90-fold, at least 95-fold, at least 100-fold, or greater relative to a lateral flow device and/or an assay that comprises colloidal gold without platinum.

In certain embodiments, the lateral flow devices and/or the assays disclosed herein and throughout have a specificity that is least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least at least 75-fold, at least 80-fold, at least 85-fold, at least 90-fold, at least 95-fold, at least 100-fold, or greater than one or more of QuickVue™, Ellume™, BD Veritor System™, BinaxNow™, PanBio™, and AccessBio™ detection devices and/or assays.

In certain embodiments, the lateral flow device and/or the assay disclosed herein and throughout has a limit of detection that is least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least 40-fold lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least 95-fold lower, at least 100-fold lower, or less than a lateral flow device and/or an assay that does not comprise an antibody-Pt conjugate, an antibody-Au/Pt conjugate, or a platinum colloid core (PtC). In certain embodiments disclosed herein and throughout, limit of detection of such lateral flow devices and/or assays is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least lower, at least 30-fold lower, at least 35-fold lower, at least 40-fold lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least lower, at least 90-fold lower, at least 95-fold lower, at least 100-fold lower, or less than to a lateral flow device and/or an assay that comprises colloidal gold without platinum.

In certain embodiments, the lateral flow devices and/or the assays disclosed herein and throughout has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least 95-fold lower, at least 100-fold lower, or less than one or more of QuickVue™, Ellume™, BD Veritor System™, BinaxNow™, PanBio™, and AccessBio™ detection devices and/or assays.

The results obtained from using the lateral flow device can, for example, be used to identify subjects who have active infection and should be subjected to isolation or medical treatment. The results can also be used to identify subjects who do not have active infection and are no longer contagious.

The present disclosure provides descriptions of lateral flow devices. One skilled in the art will recognize that other embodiments of lateral flow devices are suitable for use in detecting a coronavirus infection in a subject.

In some embodiments, the lateral flow device comprises a self-contained, multi-layered structure having both absorbent and non-absorbent materials that form a solid-phase which is used as a device for conducting an immunoassay.

The liquid sample can be obtained from a human or non-human subject and is a biological liquid sample, for example blood, serum, plasma, whole blood or urine, but is preferably a nasopharyngeal sample taken using a swab. The sample may be obtained from a negative control subject who has not been infected with a coronavirus, or the liquid sample may be obtained from a subject having or suspected of having had a coronavirus infection. The subject may currently be infected, or may have recently been infected, with SARS-CoV-2 coronavirus.

### Certain Exemplary Embodiments

Embodiment 1. A lateral flow device comprising a test strip, wherein the test strip comprises:
a sample application zone,
a conjugate pad,
an antigen detection zone comprising a test line and a control line; and
an absorbent pad to drive the flow of sample and reagents from the sample application zone to the detection zones.

Embodiment 2. The lateral flow device of Embodiment 1, wherein the sample application zone comprises absorbent material and can optionally include reagents for improving or maintaining the solubility of sample components such as proteins.

Embodiment 3. The lateral flow device of Embodiment for Embodiment 2, wherein the conjugate pad comprises conjugates that comprise an antibody that specifically binds a SARS-CoV-2 antigen.

Embodiment 4. The lateral flow device of any of Embodiments 1-3, wherein the conjugate pad comprises conjugates that comprise an antibody that specifically binds a SARS-CoV-2 antigen, wherein the SARS-CoV-2 antigen is an S protein or a nucleocapsid protein.

Embodiment 5. The lateral flow device of any of Embodiments 1-4, wherein the conjugate pad comprises conjugates that comprise an antibody that is conjugated to a nanoparticle for detection of an antigen-antibody conjugate.

Embodiment 6. The lateral flow device of any of Embodiments 1-5, wherein the conjugate pad comprises conjugates that comprise an antibody conjugated to a nanoparticle for detection of an antigen-antibody conjugate, wherein the nanoparticle comprises platinum.

Embodiment 7. The lateral flow device of any of Embodiments 1-6, wherein the conjugate pad comprises conjugates that comprise an antibody conjugated to a nanoparticle for detection of an antigen-antibody conjugate, wherein the nanoparticle comprises colloidal gold/platinum particles.

Embodiment 8. The lateral flow device of any of Embodiments 1-7, wherein the conjugate pad comprises conjugates that comprise an antibody conjugated to a nanoparticle for detection of an antigen-antibody conjugate, wherein the nanoparticle comprises a bimetallic nanoparticle.

Embodiment 9. The lateral flow device of any of Embodiments 1-8, wherein the conjugate pad comprises conjugates that comprise an antibody conjugated to a nanoparticle for detection of an antigen-antibody conjugate, wherein the nanoparticle comprises a platinum (Pt)-palladium (Pd) bimetallic nanoparticle (Pt/Pd NP), a platinum (Pt)-cobalt (Co) bimetallic nanoparticle (Pt/Co NP), a platinum (Pt)-nickel (Ni) bimetallic nanoparticle (Pt/Ni NP), a platinum (Pt)-iron (Fe) bimetallic nanoparticle (Pt/Fe NP), or a platinum (Pt)-gold (Au) bimetallic nanoparticle (Pt/Au NP).

Embodiment 10. The lateral flow device of any of Embodiments 1-9, wherein the conjugate pad comprises conjugates that comprise an antibody conjugated to a nanoparticle for detection of an antigen-antibody conjugate, wherein the nanoparticle has peroxidase activity.

Embodiment 11. The lateral flow device of any of Embodiments 1-10, wherein the test line comprises pre-set immobilized capture antibodies.

Embodiment 12. The lateral flow device of any of Embodiments 1-11, wherein the test line comprises a substrate for peroxidase activity that provides a quantifiable color change.

Embodiment 13. The lateral flow device of any of Embodiments 1-12, wherein the test line comprises a substrate for peroxidase activity, wherein the substrate for peroxidase activity is 3,3′,5,5′-tetramethylbenzidine (TMB), aminoethyl carbazole (AEC), 3,3′-diaminobenzidine (DAB), or o-phenylenediamine dihydrochloride (oPD).

Embodiment 14. The lateral flow device of any of Embodiments 1-13, wherein the lateral flow device has a sensitivity that is increased least 1-fold, at least 2-fold, at least 3-fold, at least 4-fold, at least 5-fold, at least 6-fold, at least 7-fold, at least 8-fold, at least 9-fold, at least at least 15-fold, at least 20-fold, at least 25-fold, at least 30-fold, at least 35-fold, at least at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that does not comprise an antibody-Pt conjugate, an antibody-Au/Pt conjugate, or a platinum colloid core.

Embodiment 15. The lateral flow device of any of Embodiments 1-14, wherein the lateral flow device has a sensitivity that is increased least 1-fold, at least 2-fold, at least 3-fold, at least 4-fold, at least 5-fold, at least 6-fold, at least 7-fold, at least 8-fold, at least 9-fold, at least at least 15-fold, at least 20-fold, at least 25-fold, at least 30-fold, at least 35-fold, at least at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that comprises colloidal gold without platinum.

Embodiment 16. The lateral flow device of any of Embodiments 1-15, wherein the lateral flow device has a sensitivity that is least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least at least 75-fold, at least 80-fold, at least 85-fold, at least 90-fold, at least 95-fold, at least 100-fold, or greater than one or more of QuickVue™, Ellume™, BD Veritor System™, BinaxNow™, PanBio™, and AccessBio™.

Embodiment 17. The lateral flow device of any of Embodiments 1-16, wherein the lateral flow device has a specificity that is increased least 1-fold, at least 2-fold, at least 3-fold, at least 4-fold, at least 5-fold, at least 6-fold, at least 7-fold, at least 8-fold, at least 9-fold, at least at least 15-fold, at least 20-fold, at least 25-fold, at least 30-fold, at least 35-fold, at least at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that does not comprise an antibody-Pt conjugate, an antibody-Au/Pt conjugate, or a platinum colloid core.

Embodiment 18. The lateral flow device of any of Embodiments 1-17, wherein the lateral flow device has a specificity that is increased least 1-fold, at least 2-fold, at least 3-fold, at least 4-fold, at least 5-fold, at least 6-fold, at least 7-fold, at least 8-fold, at least 9-fold, at least at least 15-fold, at least 20-fold, at least 25-fold, at least 30-fold, at least 35-fold, at least at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that comprises colloidal gold without platinum.

Embodiment 19. The lateral flow device of any of Embodiments 1-18, wherein the lateral flow device has a specificity that is least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least at least 75-fold, at least 80-fold, at least 85-fold, at least 90-fold, at least 95-fold, at least 100-fold, or greater than one or more of QuickVue™, Ellume™, BD Veritor System™, BinaxNow™, PanBio™, and AccessBio™.

Embodiment 20. The lateral flow device of any of Embodiments 1-19, wherein the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least 95-fold lower, at least 100-fold lower, or less than a lateral flow device and/or an assay that does not comprise an antibody-Pt conjugate, an antibody-Au/Pt conjugate, or a platinum colloid core (PtC).

Embodiment 21. The lateral flow device of any of Embodiments 1-20, wherein the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least 95-fold lower, at least 100-fold lower, or less than a lateral flow device and/or an assay that comprises colloidal gold without platinum.

Embodiment 22. The lateral flow device of any of Embodiments 1-21, wherein the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least 95-fold lower, at least 100-fold lower, or less than one or more of QuickVue™, Ellume™, BD Veritor System™, BinaxNow™, PanBio™, and AccessBio™ detection devices and/or assays.

Embodiment 23. A kit comprising a lateral flow device of any of Embodiments 1-22 and one or more of: one or more swabs, one or more pouches or bottles comprising a sample buffer, one or more sample tubes, one or more droppers, and one or more pipetting devices.

Embodiment 24. A kit comprising a lateral flow device of any of Embodiments 1-22 and one or more of: one or more swabs, one or more pouches or bottles comprising a sample buffer, one or more sample tubes, one or more droppers, one or more pipetting devices, and a colorimetric substrate for peroxidase activity.

Embodiment 25. A method for detecting in a liquid sample from a subject the presence or absence of a SARS-Cov-2 protein, the method comprising the steps of:
a) providing a liquid sample from the subject;
b) dispensing the liquid sample onto the sample application zone of a lateral flow device of any one of the embodiments provided herein under a condition that is suitable for lateral flow of the liquid sample and soluble proteins contained therein, wherein the lateral flow moves the liquid sample from the sample application zone through the conjugate pad of a test strip, through the detection zone, and through the absorbent pad, where a SARS-CoV-2 protein present in the sample binds to Au/Pt nanoparticle antibody conjugates in the conjugate pad to form SARS-CoV-2 protein-Au/Pt nanoparticle antibody conjugates complexes, and wherein the SARS-CoV-2 protein-Au/Pt nanoparticle antibody conjugate complexes are able to migrate to the test line of the test strip.

Embodiment 26. The method of Embodiment 25, wherein the SARS-Cov-2 protein is a nucleocapsid (N) protein.

Embodiment 27. The method of Embodiment 25 or Embodiment 26, the method further comprising:
c) detecting a signal at the control line;
d) adding a substrate for a peroxidase at the test line; and
e) observing a colorimetric reaction at the test line.

### EXAMPLES

### Example 1. An Exemplary COVID-19 Antigen Rapid Test Cassette

The exemplary COVID-19 Antigen Rapid Test or COVI-STIX™ Cassette described in this Example is a lateral flow immunoassay for the detection of the nucleoprotein of SARS-CoV-2 in a sample taken from a nasopharyngeal (NP) swab. It can provide a preliminary test result to aid in the diagnosis of infection with SARS-CoV-2 virus. The test can be performed within 15-20 minutes by minimally skilled personnel without the use of laboratory equipment. Interpretation or use of this test result should be based on comprehensive clinical and other laboratory information as well as on the professional judgment of health care providers. Alternative test method(s) may be considered to confirm the test result obtained by this test.

The COVI-STIX™ test is a lateral flow chromatographic immunoassay. The test strip in the cassette consists of: a black colored conjugate pad containing mouse anti-SARS-CoV-2 nucleoprotein monoclonal antibodies conjugated with colloidal gold/platinum (Au/Pt); and a nitrocellulose membrane strip containing a test line and a control line. In this exemplary COVI-STIX lateral flow device, the test line was pre-coated with mouse monoclonal antibodies specific for SARS-CoV-2 nucleoprotein, and the control line was pre-coated with anti-mouse IgG polyclonal antibodies as internal controls for the operation of the test strip.

When an adequate volume of test specimen is dispensed into the sample well of the test cassette, the specimen migrates by capillary action along the test strip. The SARS-CoV-2 nucleocapsid protein, if present in the specimen, will bind to the mouse antibody-Au/Pt conjugates in the conjugate pad. The immunocomplex is then captured by the mouse anti-nucleoprotein monoclonal antibody in the test line zone, forming a black colored test line, indicating a SARS-CoV-2 virus positive test result indicative of infection with the virus.

Absence of a visible test line suggests a negative result. Each test contains an internal control at the control line which should exhibit a black colored line of the control antibodies regardless of color development on any of the test lines. If the control line does not develop, the test result is invalid and the sample must be retested with another device.

The test kit provides individually sealed foil pouches containing: one cassette device, a dessicant, sample tubes (25), lysis buffer (10 ml), sterile nasopharyngeal swabs (25), and instructions. The user is advised that the test is for in vitro diagnostic use. The kit may be stored at temperatures between 2° C. and 30° C. If stored at 2-8° C., the test device is to be brought to room temperature before opening. The test device is stable until the expiration date printed on the sealed pouch.

To use the kit, collect a nasopharyngeal swab sample by carefully inserting the swab provided in the kit into the nostril or pharynx that presents the most secretion under visual inspection. Using gentle rotation, push the swab until resistance is met at the level of the turbinate. Rotate the swab several times against the nostril or pharynx wall then remove it from the nostril or pharynx. The swab in inserted into a sample tube into which 0.3 ml (about 10 drops) of provided Lysis Buffer has been added. The swab is then rotated at least 6 times while pressing the swab end against the bottom and side of the sample tube. After 1-3 minutes, the tube is squeezed several times from the outside walls to immerse the swab, after which the swab is removed, and the extracted sample remains in the tube.

Specimens should be tested as soon as possible after collection. The sample and test components should be brought to room temperature before use. The test device is removed from its pouch and placed on a clean flat surface. The cap of the sample tube, which includes a dropper type attachment, is used to distribute the sample (3 to 4 drops, or approximately 100 μl) into the sample well of the device, avoiding air bubbles.

In some test kits, at least five minutes after the sample has been applied, from one to three drops of a peroxidase substrate reagent provided with the kit is added to the test line. According to some protocols, the peroxidase substrate reagent is added after the development of a signal at the control line. The test result can be read within 15 minutes of loading the sample. After 20 minutes, results are considered unreliable and the test should be repeated. If the C line does not develop, the test is invalid and must be repeated with a new device.

When interpretating the assay result: if only the C line is visible, the absence of a colored line in the Test Line zone indicates that SARS-Co-2 is not detected. This is a negative test result. In an alternative situation, if in addition to the C line, a T line appears, the test indicates that SARS-CoV-2 is detected. This is a positive test result. It is advised that subjects having a positive result have the results confirmed with another type of test prior to diagnosis.

### Example 2. Preparation of Antibody-Au/Pt NP Conjugates

To generate antibody-gold/platinum nanoparticles (Au/Pt Nps), the anti-SARS-CoV-2 antibody (e.g., anti-nucleocapsid protein antibody) can be mixed with Pt/Au nanoparticles (NPs), followed by gentle shaking for 1 h at room temperature. BSA is added to a final concentration of 1% BSA, and the mixture is incubated for 30 min. After washing with PBS/1% BSA, the preparation is centrifuged at 8000 rpm for 10 min. The supernatant is removed and the prepared Pt/Au NP-antibody conjugates are then suspended in a one tenth volume (relative to the original product mixture) of 10 mM PBS, 0.25% Tween-20, 10% sucrose, and 5% BSA, pH 7.4.

In some embodiments metal precursors can be reduced by a strong reducing agent in liquid phase to form faceted crystal morphology, which is one way to synthesize metallic alloy nanostructures. Additionally, self-assembly of surfactants into spherical micelles can employ faceted crystal as templates to synthesize porous metal NPs. The resulting bimetallic porous Pt/Au nanocolloids provide high surface area with multiple activity sites on the concave surface.

The average size of NPs may be for example from about 5 nm to about 500 nm, or from about 20 nm to about 100 nm, such as about 50 nm. Fine Pt particles may form several branched structures on the surface of the Au/Pt NPs forming a porous structure. Pt/Au NPs can have a catalytic peroxidase activity that can be assessed, for example, using colorimetric tests (e.g., using TMB-H₂O₂ substrate. The sensitivity of the colorimetric assay can be significantly higher that with PT nanopowder. See for example, US 2016/0349249 and US 2017/0336398, incorporated herein by reference in their entireties. Detailed methods for producing Pt nanoparticles, Au/Pt nanoparticles and Au/Pt nanoparticle-antibody conjugates are also disclosed in US 2016/0349249 and US 2017/0336398.

For use in assays, the Au/Pt NP-antibody conjugates (specifically binding a SARS-CoV antigen, e.g., the nucleocapsid (N) protein of SARS-CoV-2).

An assay strip that includes the Au/Pt NP-antibody conjugates in the conjugate pad can optionally also include a colorometric peroxidase reagent, for example, TMB, AEC, DAB, or oPD in the test line zone for detection of the positive signal. In such assays, the migration of the sample, and, if present, SARS-CoV-2 nucleocapsid protein, through the test strip by capillary force with enable the nucleocapsid protein to bind the Pt/Au NPs in the conjugation pad. The complexes will then pass along through the nitrocellulose membrane by capillary action until reaching the capture antibody which binds the nucleocapside protein in the test line to achieve the sandwich immunoreaction. If the test line does not also include a peroxidase substrate, a substrate such as TMB or AEC may be added to the test line zone, preferable after a signal is observed in the control line, demonstrating that the complexes have migrated through the strip. The final signal intensity on test line can be observed and optionally quantified using a test strip reader, such as a portable fluorescence strip reader e.g., ESE-Quant GOLD (DCN Inc.; Irvine, Calif.).

### Example 3. COVI-STIX/VTM Lysis Buffer Swab Assay Performance

The objective of this study was to use the COVI-STIX assay (Lateral Flow technology) to conduct a clinical evaluation of dry frozen nasopharyngeal (NP) Swabs resuspended in 200 uL Viral Transfer Media (VTM) from a randomized set of specimens performed blinded to support an Emergency Use Authorization (EUA).

Design Input/Product Requirements Covered

From the FDA's EUA Antigen Template (version Oct. 26, 2020), the clinical evaluation study should meet or exceed the following acceptance criteria: “A minimum of 95% positive and negative agreement” (based on proposed test design with a minimum of 30 individual natural positive clinical specimens according to an authorized EUA RT-PCR method and a minimum of 30 individual natural negative specimens).

The exemplary COVI-STIX device employed in this Example comprised a lateral flow immunoassay that used a porous platinum nanocatalyst core (PtNC or PnC, used interchangeably herein and throughout) which yields up to 100-fold increases in sensitivity over conventional lateral flow colloidal gold assays. If viral antigen is present, the sample will encounter the PtNC particles targeted specifically to the viral nucleocapsid (N) antigen. The complex will then be captured in a strong biotin-avidin complex, producing a positive line on the membrane stick.

Nasopharyngeal swabs were collected and then placed into empty tubes and frozen for transport. Each sample was then thawed and placed into 200 uL of VTM to remove sample from the swab. After removal, the resulting liquid was mixed with Lysis Buffer and transferred to the sample well of the COVI-STIX cassette. Capillary action moved the sample contents through the reagents built into the device. In less than 15 minutes a noticeable line appeared to indicate a positive result. The device also contained a control line to indicate that the device was working properly.

Sample Size

Fifty samples were tested based on the need to exceed the EUA clinical evaluation. The FDA's EUA template requires a minimum of 30 individual natural positive specimens and a minimum of 30 individual natural negative specimens in order to reflect a realistic range of Ct values in the positive samples.

Statistical Analysis

Following the completion and documentation of the COVI-STIX test results (positive/negative), the comparator results were unblinded. The results were tabulated into a 4×4 table as provided in Table 1, below, along with two-sided 95% confidence intervals (Score Method) for the positive percent agreement (PPA) and the negative percent agreement (NPA).

TABLE 1

| | EUA qRT-PCR+ | EUA qRT-PCR− | Subtotal/Total |
|---|---|---|---|
| COVI-TRACE+ | True Pos “A” | False Pos “B” | A + B |
| COVI-TRACE− | False Neg “C” | True Neg “D” | C + D |
| Subtotal/Total | A + C | B + D | A + B + C + D |

PPA=(A)/(A+C) 95% CI (x.x, x.x)
NPA=(D)/(B+D) 95% CI (x.x, x.x)

These values were compared to the acceptance criteria and provided in a line listing including any justifications for exclusion of results.

Validity of the study was evaluated based on the consistency of the positive and negative controls consisting of recombinant N antigen or a known positive patient sample (positive) and VTM buffer (negative). Each day of testing was required to have one correct positive and one correct negative control along with each new batch of reagents. Furthermore, PPA and NPA was required to be greater than or equal to 95%.

Test Preparations and Procedure

The swab was in a 1.5 mL Eppendorf tube containing 200 uL of VTM buffer. Using mechanical agitation, swab was swirled and spun to mix well into the VTM. The swab was then discarded. Approximately 60 uL of the sample was then transferred to a fresh tube containing of the COVI-STIX lysis buffer, mixed gently, and allowed to incubate at room temperature for approximately three minutes. Using a transfer pipet, approximately 100 uL of the mixture was applied into the sample receiving well of the COVI-STIX cassette and allowed to absorb into the cassette for approximately 15 minutes for the sample via capillary action. Results were then recorded after 20 minutes by photographing the cassettes in rack to preserve line intensity.

Results

A total of 105 nasopharyngeal samples were tested. The swab samples were collected at clinical sites and placed in sterile tubes and frozen until thawed for testing. Unbeknownst to the operators, there were 55 PCR-positive samples and 50 negative samples. In this VTM-lysis buffer method, the dry frozen-thawed swabs were first immersed in a volume of 200 uL of VTM. After mechanical agitation, 60 uL of the sample was pipetted into 60 uL of the COVI-STIX Lysis Buffer for at least 3 minutes. This was then pipetted to the sample receiving well of the cassette. Samples were mixed together randomly and operators were not aware of which samples were positive and negative and the cassettes were read by at least two operators and photographed.

For patients presenting with symptoms 0-7 days before collection, NP swab results demonstrated that of the 51 unmasked positives by RT-PCR, 50 were scored as positive by COVI-STIX while one was scored as negative by COVI-STIX, yielding a sensitivity, or PPA, of 98.04% (95% CI=89.70% to 99.65%). The one COVI-STIX negative sample had RT-PCR Ct values (ORF/N/S genes respectively) of 29/29/30. The Specificity, or NPA, of this group was 100%: all 50 negatives by RT-PCR scored as negative by COVI-STIX. These results are tabulated in Table 2, below:

TABLE 2
Summary of positive and negative agreement with real patient
specimen type (0-7 days infection before collection)

| | | | PCR-confirmed |
|---|---|---|---|
| | COVISTIX | COVISTIX | samples |
| Test result | Positive | Negative | Total |
| PCR Positive | 50 | 1 | 51 |
| PCR Negative | 0 | 50 | 50 |

| | |
|---|---|
| Positive Percent | 98.04% (95% CI 89.70% to 99.65%) |
| Agreement | |
| Negative Percent | 100% (95% CI 92.87% to 100.00%) |
| Agreement | |

Sensitivity is the Positive Percent Agreement (PPA), or true positive/(true positive + false negative)
Specificity is the Negative Percent Agreement (NPA), or true negative/(true negative + false positives)

For patients presenting with symptoms 0-14 days before collection, NP swab results demonstrated that of the 55 unmasked positives by RT-PCR, 53 were scored as positive by COVI-STIX while two were scored as negative by COVI-STIX, yielding a sensitivity, or PPA, of 96.36% (95% CI=87.68% to 99.00%). The two COVI-STIX negative samples had RT-PCR Ct values of ORF/N/S genes of 29/29/30; and 29/26/30, respectively. The specificity, or NPA, of this group was 100%: all 50 negatives by RT-PCR scored as negative by COVI-STIX. These results are tabulated in Table 3, below:

TABLE 3
Summary of positive and negative agreement with real patient
specimen type (0-14 days infection before collection)

| | | | PCR-confirmed |
|---|---|---|---|
| | COVISTIX | COVISTIX | samples |
| Test result | Positive | Negative | Total |
| PCR Positive | 53 | 2 | 55 |
| PCR Negative | 0 | 50 | 50 |

| | |
|---|---|
| Positive Percent | 96.36% (95% CI 87.68% to 99.00%) |
| Agreement | |
| Negative Percent | 100% (95% CI 92.87% to 100.00%) |
| Agreement | |

Sensitivity is the Positive Percent Agreement (PPA), or true positive/(true positive + false negative)
Specificity is the Negative Percent Agreement (NPA), or true negative/(true negative + false Positives)

### Example 4. COVI-STIX VTM/Lysis Buffer Nasal (Shallow) Swab Assay Performance

The objective of this study was to use the COVI-STIX assay (Lateral Flow technology) to conduct a clinical evaluation of dry frozen nasal (N, “shallow”) swabs resuspended in 200 uL Viral Transfer Media (VTM) from a randomized set of specimens performed blinded to support an Emergency Use Authorization (EUA).

Design Input/Product Requirements Covered

From the FDA's EUA Antigen Template (version Oct. 26, 2020), the clinical evaluation study should meet or exceed the following acceptance criteria: “A minimum of 95% positive and negative agreement” (based on proposed test design with a minimum of 30 individual natural positive clinical specimens according to an authorized EUA RT-PCR method and a minimum of 30 individual natural negative specimens).

The exemplary COVI-STIX device employed in this Example comprised a lateral flow immunoassay that used a porous platinum nanocatalyst core (PtNC or PnC, used interchangeably herein and throughout) which yields up to 100-fold increases in sensitivity over conventional lateral flow colloidal gold assays. If viral antigen is present, the sample will encounter the PtNC particles targeted specifically to the viral nucleocapsid (N) antigen. The complex will then be captured in a strong biotin-avidin complex, producing a positive line on the membrane stick.

Nasal swabs were collected and then placed into an empty tube and frozen for transport. Each sample was then thawed and placed into 200 uL of VTM to remove sample from the swab. After removal, the resulting liquid was mixed with Lysis Buffer and transferred to the sample well of the COVI-STIX cassette. Capillary action moved the sample contents through the reagents built into the device. In less than 15 minutes a noticeable line appeared to indicate a positive result. The device also contained a control line to indicate that the device was working properly.

Sample Size

Fifty samples were tested based on the need to exceed the EUA clinical evaluation. The FDA's EUA template requires a minimum of 30 individual natural positive specimens and a minimum of 30 individual natural negative specimens in order to reflect a realistic range of Ct values in the positive samples.

Statistical Analysis

Following the completion and documentation of the COVI-STIX test results (positive/negative), the comparator results were unblinded. The results were tabulated into a 4×4 table as provided in Table 1, above, along with two-sided 95% confidence intervals (Score Method) for the positive percent agreement (PPA) and the negative percent agreement (NPA). These values were compared to the acceptance criteria and provided in a line listing including any justifications for exclusion of results.

Validity of the study was evaluated based on the consistency of the positive and negative controls consisting of recombinant N antigen or a known positive patient sample (positive) and VTM buffer (negative). Each day of testing was required to have one correct positive and one correct negative control along with each new batch of reagents. Furthermore, PPA and NPA was required to be greater than or equal to 95%.

Test Preparations and Procedure

The swab was in a 1.5 mL Eppendorf tube containing 200 uL of VTM buffer. Using mechanical agitation, swab was swirled and spun to mix well into the VTM. The swab was then discarded. Approximately 60 uL of the sample was then transferred to a fresh tube containing 60 uL of the COVI-STIX lysis buffer, mixed gently, and allowed to incubate at room temperature for approximately three minutes. Using a transfer pipet, approximately 100 uL of the mixture was applied into the sample receiving well of the COVI-STIX cassette and allowed to absorb into the cassette for approximately 15 minutes for the sample via capillary action. Results were then recorded after 20 minutes by photographing the cassettes in rack to preserve line intensity.

Results

A total of 100 nasal swab samples were tested. The swab samples were collected at clinical sites and placed in sterile tubes and frozen until thawed for testing. Unbeknownst to the operators, there were 50 PCR-positive samples and 50 negative samples. In this VTM-lysis buffer method, the dry frozen-thawed swabs were first immersed in a volume of 200 uL of VTM. After mechanical agitation, 60 uL of the sample was pipetted into 60 uL of the COVI-STIX Lysis Buffer for at least 3 minutes. This was then pipetted to the sample receiving well of the cassette. Samples were mixed together randomly and operators were not aware of which samples were positive and negative and the cassettes were read by at least two operators and photographed. Samples were obtained from patients who presented with symptoms 0-9 days before collection.

During the course of the analysis of this study, some samples were re-submitted for a confirmatory PCR. ID #'s 832 and 849 resulted in PCR results that changed from an initial negative to positive. Separate analyses—one excluding these two samples and the other including them as their re-PCR results—are provided below.

Excluding #'s 832 and 849, the results showed that of the 50 unmasked positives, 47 were scored as positive while three were scored as negative, yielding a sensitivity (PPA) of 94.00% (95% CI=83.78% to 97.94%). Including #'s 832 and 849 as positive PCR samples, the total number of PCR positives=52, with 48 scored as positive and 4 scored as negative yielding a PPA of 92.31% (95% CI: 81.83% to 96.97%). The results also showed that of the 48 unmasked negatives, 46 were scored as negative yielding a specificity (NPA) of 95.83% (95% CI=86.02% to 98.85%).

As presented in Tables 4 and 5 below, the COVI-STIX negative results of some of the RT-PCR positive samples may have been reasonable given that there were relatively high RT-PCR Ct values: For example, Sample #849 (N2 Ct=30.13 on repeat RT-PCR), #792 (N2 Ct=32.05), #771 (N2 Ct=38.52) and #797 (Ct=39.93).

TABLE 4
Summary of positive and negative agreement with patient
samples without ID #832 and #849

| | qPCR confirmed samples | | |
|---|---|---|---|
| Test result | Positive | Negative | Total |
| Positive | 47 | 3 | 50 |
| Negative | 2 | 46 | 48 |

| | |
|---|---|
| Positive | 94.00% (95% CI: 83.78% to 97.94%) |
| agreement | |
| Negative | 95.83% (95% CI: 86.02% to 98.85%) |
| agreement | |

TABLE 5
Summary of positive and negative agreement with
patient samples with ID# 832 and 849

| | qPCR confirmed samples | | |
|---|---|---|---|
| Test result | Positive | Negative | Total |
| Positive | 48 | 4 | 52 |
| Negative | 2 | 46 | 48 |

| | |
|---|---|
| Positive | 92.31% (95% CI: 81.83% to 96.97%) |
| agreement | |
| Negative | 95.83% (95% CI: 86.02% to 98.85% |
| agreement | |

### Example 5. COVI-STIX VTM/Lysis Buffer Nasopharyngeal Independent Field Study

A field study was independently performed by Instituto de Diagnóstico y Referencia Epidemiológicos (InDRE Study) to assess sensitivity and specificity of an exemplary COVI-Stix lateral flow device.

Sample Size

Samples were obtained from 465 symptomatic patients who experienced symptoms for 0-7 days prior to obtaining samples. For all symptomatic patients, at the time of collection, results showed that of the 71 unmasked positives by RT-PCR, 68 were scored as positive by COVI-STIX, while 3 were scored as negative by COVI-STIX, yielding a sensitivity (PPA) of (95% CI: 87.99% to 98.59%). For all patients that were negative (394) by RT-PCR, 388 were scored as negative by COVI-STIX and 6 were scored as positive by COVI-STIX, resulting in a specificity (NPA) of 98.48% (95% CI: 96.78% to 99.29%). The results are summarized in Table 6, below:

TABLE 6
Summary of positive and negative agreement with patient samples

| | COVISTIX | COVISTIX | qPCR-confirmed samples |
|---|---|---|---|
| Test result | Positive | Negative | Total |
| PCR Positive | 68 | 3 | 71 |
| PCR Negative | 6 | 388 | 394 |

| | |
|---|---|
| Positive Percent | 95.77% (95% CI: 87.99% to 98.59%) |
| Agreement | |
| Negative Percent | 98.48% (95% CI: 96.78% to 99.29%) |
| Agreement | |

Sensitivity is called as Positive Percent Agreement (PPA), or true positive/(true positive+false negative)
Specificity is called as negative percent agreement (NPA), or true negative/(true negative+false Positives)

### Example 6: Side by Side Comparison of Exemplary COVI-STIX Lateral Flow Device to SD Biosensor

A side-by-side comparison was performed between the STANDARD Q test (SD Biosensor Lateral Flow COVID-19 Ag test) and an exemplary COVI-STIX lateral flow device immunoassay test. Using a single set of serially diluted inactivated SARS-CoV-2 virus standard with known TCID50, the Limit of Detection (LoD) was determined for each test. Results demonstrated that the COVI-STIX test was 8-fold more sensitive (LoD of 7.8 TCID50/mL) than the STANDARD Q test (62.5 TCID50/mL).

### Example 7: PATH Biorepository Blinded Dilution Panel Study

The PATH developed SARS-CoV-2 Clinical Dilution Panel is offered as 17 unique panel members of 130 μL for the purpose of running with rapid diagnostic tests (RDTs) designed to detect SARS-CoV-2 virus protein antigens from swabs which are eluted into pre-measured buffer at the time of testing. This Example outlines the evaluation of the PATH Clinical Dilution Panel on COVISTIX. The vials are labeled with Dilution Level 0, and Dilution Levels 2-17; Dilution Level 1 was not used, and Dilution 0 was a negative control.

The results, provided in Table 7 below, demonstrated that COVISTIX lateral flow device was sensitive enough to detect analyte in Dilutions 2 through 15 of the PATH Clinical Dilution Panel. Dilution 15 had the closest analyte concentration equivalent to Limit of Detection test line intensity. When unblinded, level 15 represented a Ct value of 28.1, CDC 2019-nCoV Real Time RT-PCR N1 assay, viral count/ml of 2.54×10⁴, and an N antigen protein concentration of 0.091 ng/ml.

TABLE 7

| Clinical Dilution | CDC 2019-nCov Real-Time RT- | Ct | N antigen protein |
|---|---|---|---|
| Level ID | PCR N1 assay, viral count/mL | value | concentration, ng/mL |
| 0 | negative | — | Not detected (<LLOQ) |
| 2 | 7.66E+07 | 17.2 | 225.995 |
| 3 | 3.48E+07 | 18.1 | 116.765 |
| 4 | 1.54E+07 | 19.3 | 52.954 |
| 5 | 6.51E+06 | 20.4 | 30.692 |
| 6 | 3.41E+06 | 21.4 | 18.214 |
| 7 | 2.40E+06 | 21.8 | 8.423 |
| 8 | 1.43E+06 | 22.7 | 4.600 |
| 9 | 1.27E+06 | 22.7 | 2.171 |
| 10 | 9.53E+05 | 23.1 | 1.809 |
| 11 | 4.11E+05 | 24.3 | 0.882 |
| 12 | 2.01E+05 | 25.2 | 0.537 |
| 13 | 6.65E+04 | 26.8 | 0.258 |
| 14 | 4.27E+04 | 27.3 | 0.145 |
| 15 | 2.54E+04 | 28.1 | 0.091 |
| 16 | 1.25E+04 | 29.1 | 0.057 |
| 17 | 8.67E+03 | 29.5 | 0.026 |

### Example 8: Exemplary COVISTIX Lateral Flow Device PATH Biorepository Clinical Dilution Panel 2 (Levels 23-38 Evaluation)

A study similar to that disclosed in Example 7 above was performed to assess whether COVISTIX™ was sensitive enough to detect analyte in samples prepared from Dilution Levels 24 through 31 of PATH Clinical Dilution Panel. Based on test line intensity, the samples prepared from Dilution Level 31 has the closest analyte concentration equivalent to Limit of Detection. According to PATH Clinical Dilution Panel Results unblinding document, Dilution 31 (Result≈1×LoD 25 TCID₅₀/mL of the KMD native inactivated whole virus control) contained 115 pg/mL N Ag. After dilution 1:3 with lysis buffer, the concentration actually applied to cassette was 38.3 pg/mL. N1 genome equivalent is (2.37E+4)/mL.

### Example 9: Exemplary COVISTIX Lateral Flow Device Performance Versus Abbott BinaxNOW™ Inactivated Whole Virus: Limit of Detection (LoD) Comparison

The purpose of this Example was, inter alia, to compare the LoD performance of COVISTIX™ versus Abbott's BinaxNOW™ using a native inactivated SARS-CoV-2 whole virus. A native inactivated SARS-CoV-2 whole virus product from KMD Bioscience known to be detectable by COVISTIX™ was used. The sensitivity of each product's test strip was evaluated by conducting a direct LoD determination i.e., the exact same concentration of analyte was be applied to both test strips.

Abbott's BinaxNOW™ Load Volume Determination: COVISTIX™ procedure calls for extraction of the swab sample in about 200 μL (— 10 drops depending on dropper's drop volume) of lysis buffer in a sample extraction tube, and then application of about 100 μL (˜4 drops depending on dropper's drop volume) into sample well of cassette using the sample tube dropper cap, or alternatively with a transfer/micropipettte.

BinaxNOW™ procedure calls for extraction of the swab sample in 6 drops of extraction reagent in the test card swab well, and then application of the sample by pressing the soaked swab in the total extraction volume directly against the test strip by closing the card.

To ensure that the same analyte concentration is delivered to the test strips of both COVISTIX™ and BinaxNOW™ for a fair comparison, the BinaxNOW™ exact load volume needed to be determined. BinaxNOW COVID-19 Ag Card IFU IN195000:
Rev.1 2020/08 states LoD is 22.5 TCID₅₀/swab
Rev.2 2020/12 states LoD is 140.6 TCID₅₀/mL.

The IFU stated that the LoD determination was done by using contrived nasal swab samples that were prepared by absorbing 20 μL of each virus dilution onto the swab. Assuming that 140.6 TCID₅₀/mL was the concentration of the sample post-extraction from the swab, the total volume dispensed into the swab well was calculated to be approximately: (22.5÷140.6) mL/swab=0.160 mL/swab=160 μL/swab. At six drops per sample, this meant that the drop size should be around 26.7 μL/drop.

A drop volume study was done on the BinaxNOW™ Extraction Reagent bottle to corroborate total sample/drop volume calculation:
8 drops=186 μL i.e., 23.25 μL/drop
6 drops=168 μL i.e., 28 μL/drop
Overall average is 25.3 μL/drop
   Assuming that 140.6 TCID₅₀/mL was the concentration of the sample before applying 20 μL onto the swab, that meant that only (140.6×0.02) TCID₅₀/swab=2.81 TCID₅₀ was applied on the swab, which was not consistent with IFU Rev. 1. Therefore, calculations were instead based on an assumption that 140.6 TCID₅₀/mL was the concentration of the sample post-extraction from the swab.

The results, outlined in Table 8 below, demonstrated, inter alia, that the COVISTIX™ test strip was about four times more sensitive to native inactivated SARS-CoV-2 whole virus than Abbott's BinaxNOW™ test strip. Even when test lines for both products at the same control analyte concentration were visible, the clarity of the BinaxNOW™ test and control lines were not as distinct as COVISTIX™'s, possibly leading to inaccurate interpretation or lack of confidence from end users when using BinaxNOW™ test strips.

TABLE 8
LoD Comparison Results

| | LFIA Product | | | |
|---|---|---|---|---|
| | COVISTIX ™ | | BinaxNOW ™ | |
| | Volume Loaded (μL) | | | |
| | 100 | | 160 | |
| [Control] | Total loaded | | Total loaded | |
| (TCID₅₀/mL) | (TCID₅₀) | Result | (TCID₅₀) | Result |
| 10000 | 1000 | Positive | 1600 | Positive |
| 1000 | 100 | Positive | 160 | Positive |
| 200 | 20 | Positive | 32 | Positive |
| 100 | 10 | Positive | 16 | LoD Positive |
| 50 | 5 | Positive | 8 | Negative |
| 25 | 2.5 | LoD Positive | 4 | Negative |
| 12.5 | 1.25 | Negative | 2 | Negative |

### Example 10: Comparison of COVISTIX Versus Panbio™ in Detecting Recombinant SARS-CoV2 N Antigen Variants of Concern (VoC)

This Example provides the performance of an exemplary COVI-STIX lateral flow device for detecting each of five recombinant nucleocapsid (N) protein antigens from the main SARS-CoV-2 VoCs (i.e., wild type (“Washington” or “Wuhan”), alpha (“United Kingdom”), beta (“South Africa”), gamma (“Brazil”), and delta (“India”), (obtained from Sino Biological) in comparison to the performance of a commercially available lot of Panbio™.

The results demonstrated that the Limit of Detection (LoD) of Wild Type (WT) recombinant nucleocapsid antigen (rN Ag) on COVISTIX™ was determined to be 100 pg/mL. With the wild type, even 1000 pg/mL (10×LoD of WT rN Ag on COVISTIX™) on Panbio™ was only barely visible compared to a clearly visible result with COVISTIX™. This suggested that the COVISTIX™ test cassette was about 10 times more sensitive than Panbio™'s for the original wild type strain. Additionally, all five of the tested variants were clearly detectable on COVISTIX™ at 300 pg/mL (3×LoD of WT rN Ag on COVISTIX™). In contrast, all five variants were not clearly detectable on Panbio™ at 300 pg/mL. The five variants were clearly detectable on Panbio™ at 3 ng/mL (30×LoD of WT rN Ag on COVISTIX™).

### Example 11: Comparison of Performance of an Exemplary COVI-STIX Lateral Flow Device Versus QuickVue™, Ellume™, BD Veritor System™, BinaxNow™, PanBio™, and AccessBio™ Products

A comparative analysis was performed in a manner similar to that provided in Examples 9 and 10, in which the performance of an exemplary COVI-STIX lateral flow device was compared to that observed using each of QuickVue™, Ellume™, BD Veritor System™, BinaxNow™, PanBio™, and AccessBio™ products. The results, depicted in Table 9, demonstrated that the exemplary COVI-STIX lateral flow device had the lowest LoD in comparison to all of the other products tested.

TABLE 9

| Name | 1000 PFU | 500 PFU | Comments |
|---|---|---|---|
| COVISTIX | Positive 5/5 | Visible 2/5 | N/A |
| | (100%) | (40%) | |
| Panbio | Negative 5/5 | Negative 5/5 | N/A |
| (Abbott) | (0%) | (0%) | |
| BinaxNOW | Visible 1/5 | Negative 5/5 | N/A |
| (Abbott) | (20%) | (0%) | |
| Quickvue | Visible 3/5 | Negative 5/5 | N/A |
| (Quidel) | (60%) | (0%) | |
| BD Veritor | Not tested | Not tested | Detection limit |
| | | | was >10,000 PFU |
| Ellume | Negative 5/5 | Not tested | No photo, results were |
| | (0%) | | sent via e-mail |
| AccessBio | Negative 5/5 | Not tested | N/A |
| | (0%) | | |

Throughout this application various publications, patents, and/or patent applications are referenced. The disclosures of the publications, patents and/or patent applications are hereby incorporated by reference in their entireties into this application in order to more fully describe the state of the art to which this disclosure pertains. To the extent any material incorporated by reference conflicts with the express content of this application, the express content controls.

1. A lateral flow device comprising a test strip, wherein the test strip comprises:
a sample application zone,
a conjugate pad,
an antigen detection zone comprising a test line and a control line; and
an absorbent pad to drive the flow of sample and reagents from the sample application zone to the detection zones.

2. The lateral flow device of claim 1, wherein the sample application zone comprises absorbent material and can optionally include reagents for improving or maintaining the solubility of sample components such as proteins.

3. The lateral flow device of claim 1 or claim 2, wherein the conjugate pad comprises conjugates that comprise an antibody that specifically binds a SARS-CoV-2 antigen.

4. The lateral flow device of any of claims 1-3, wherein the conjugate pad comprises conjugates that comprise an antibody that specifically binds a SARS-CoV-2 antigen, wherein the SARS-CoV-2 antigen is an S protein or a nucleocapsid protein.

5. The lateral flow device of any of claims 1-4, wherein the conjugate pad comprises conjugates that comprise an antibody that is conjugated to a nanoparticle for detection of an antigen-antibody conjugate.

6. The lateral flow device of any of claims 1-5, wherein the conjugate pad comprises conjugates that comprise an antibody conjugated to a nanoparticle for detection of an antigen-antibody conjugate, wherein the nanoparticle comprises platinum.

7. The lateral flow device of any of claims 1-6, wherein the conjugate pad comprises conjugates that comprise an antibody conjugated to a nanoparticle for detection of an antigen-antibody conjugate, wherein the nanoparticle comprises colloidal gold/platinum particles.

8. The lateral flow device of any of claims 1-7, wherein the conjugate pad comprises conjugates that comprise an antibody conjugated to a nanoparticle for detection of an antigen-antibody conjugate, wherein the nanoparticle comprises a bimetallic nanoparticle.

9. The lateral flow device of any of claims 1-8, wherein the conjugate pad comprises conjugates that comprise an antibody conjugated to a nanoparticle for detection of an antigen-antibody conjugate, wherein the nanoparticle comprises a platinum (Pt)-palladium (Pd) bimetallic nanoparticle (Pt/Pd NP), a platinum (Pt)-cobalt (Co) bimetallic nanoparticle (Pt/Co NP), a platinum (Pt)-nickel (Ni) bimetallic nanoparticle (Pt/Ni NP), a platinum (Pt)-iron (Fe) bimetallic nanoparticle (Pt/Fe NP), or a platinum (Pt)-gold (Au) bimetallic nanoparticle (Pt/Au NP).

10. The lateral flow device of any of claims 1-9, wherein the conjugate pad comprises conjugates that comprise an antibody conjugated to a nanoparticle for detection of an antigen-antibody conjugate, wherein the nanoparticle has peroxidase activity.

11. The lateral flow device of any of claims 1-10, wherein the test line comprises pre-set immobilized capture antibodies.

12. The lateral flow device of any of claims 1-11, wherein the test line comprises a substrate for peroxidase activity that provides a quantifiable color change.

13. The lateral flow device of any of claims 1-12, wherein the test line comprises a substrate for peroxidase activity, wherein the substrate for peroxidase activity is 3,3′,5,5′-tetramethylbenzidine (TMB), aminoethyl carbazole (AEC), 3,3′-diaminobenzidine (DAB), or o-phenylenediamine dihydrochloride (oPD).

14. The lateral flow device of any of claims 1-13, wherein the lateral flow device has a sensitivity that is increased least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that does not comprise an antibody-Pt conjugate, an antibody-Au/Pt conjugate, or a platinum colloid core.

15. The lateral flow device of any of claims 1-14, wherein the lateral flow device has a sensitivity that is increased least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that comprises colloidal gold without platinum.

16. The lateral flow device of any of claims 1-15, wherein the lateral flow device has a sensitivity that is least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater than one or more of QuickVue™, Ellume™, BD Veritor System™, BinaxNow™, PanBio™, and AccessBio™.

17. The lateral flow device of any of claims 1-16, wherein the lateral flow device has a specificity that is increased least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that does not comprise an antibody-Pt conjugate, an antibody-Au/Pt conjugate, or a platinum colloid core.

18. The lateral flow device of any of claims 1-17, wherein the lateral flow device has a specificity that is increased least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater relative to a lateral flow device and/or an assay that comprises colloidal gold without platinum.

19. The lateral flow device of any of claims 1-18, wherein the lateral flow device has a specificity that is least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, 65-fold, at least 70-fold, at least 75-fold, at least 80-fold, at least 85-fold, at least 90-fold, at least 95-fold, at least 100-fold, or greater than one or more of QuickVue™, Ellume™, BD Veritor System™, BinaxNow™, PanBio™, and AccessBio™.

20. The lateral flow device of any of claims 1-19, wherein the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least 40-fold lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least 95-fold lower, at least 100-fold lower, or less than a lateral flow device and/or an assay that does not comprise an antibody-Pt conjugate, an antibody-Au/Pt conjugate, or a platinum colloid core (PtC).

21. The lateral flow device of any of claims 1-20, wherein the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least 40-fold lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least 40-fold lower, at least 100-fold lower, or less than a lateral flow device and/or an assay that comprises colloidal gold without platinum.

22. The lateral flow device of any of claims 1-21, wherein the lateral flow device has a limit of detection that is at least 1-fold lower, at least 2-fold lower, at least 3-fold lower, at least 4-fold lower, at least 5-fold lower, at least 6-fold lower, at least 7-fold lower, at least 8-fold lower, at least 9-fold lower, at least 10-fold lower, at least 15-fold lower, at least 20-fold lower, at least 25-fold lower, at least 30-fold lower, at least 35-fold lower, at least 40-fold lower, at least 45-fold lower, at least 50-fold lower, at least 55-fold lower, at least 60-fold lower, at least 65-fold lower, 65-fold lower, at least 70-fold lower, at least 75-fold lower, at least 80-fold lower, at least 85-fold lower, at least 90-fold lower, at least 95-fold lower, at least 100-fold lower, or less than one or more of QuickVue™, Ellume™, BD Veritor System™, BinaxNow™, PanBio™, and AccessBio™ detection devices and/or assays.

23. A kit comprising a lateral flow device of any of claims 1-22 and one or more of: one or more swabs, one or more pouches or bottles comprising a sample buffer, one or more sample tubes, one or more droppers, and one or more pipetting devices.

24. A kit comprising a lateral flow device of any of claims 1-22 and one or more of: one or more swabs, one or more pouches or bottles comprising a sample buffer, one or more sample tubes, one or more droppers, one or more pipetting devices, and a colorimetric substrate for peroxidase activity.

25. A method for detecting in a liquid sample from a subject the presence or absence of a SARS-Cov-2 protein, the method comprising the steps of:
a) providing a liquid sample from the subject;
b) dispensing the liquid sample onto the sample application zone of a lateral flow device of any one of the embodiments provided herein under a condition that is suitable for lateral flow of the liquid sample and soluble proteins contained therein, wherein the lateral flow moves the liquid sample from the sample application zone through the conjugate pad of a test strip, through the detection zone, and through the absorbent pad, where a SARS-CoV-2 protein present in the sample binds to Au/Pt nanoparticle antibody conjugates in the conjugate pad to form SARS-CoV-2 protein-Au/Pt nanoparticle antibody conjugates complexes, and wherein the SARS-CoV-2 protein-Au/Pt nanoparticle antibody conjugate complexes are able to migrate to the test line of the test strip.

26. The method of claim 25, wherein the SARS-Cov-2 protein is a nucleocapsid (N) protein.

27. The method of claim 25 or claim 26, the method further comprising:
c) detecting a signal at the control line;
d) adding a substrate for a peroxidase at the test line; and
e) observing a colorimetric reaction at the test line.
